(19) United States
(12) Patent Application Publication
(10) Pub. No.: US 2022/0091121 A1
(43) Pub. Date: Mar. 24, 2022

(54) CANCER DETECTION UTILIZING NORMAL TISSUE ADJACENT TO BREAST TUMORS WITH GENETIC ANCESTRY-MAPPING

(71) Applicant: THE TRUSTEES OF INDIANA UNIVERSITY, Bloomington, IN (US)

(72) Inventor: Harikrishna NAKSHATRI, Zionsville, IN (US)

(21) Appl. No.: 17/298,234

(22) PCT Filed: Nov. 25, 2019
(86) PCT No.: PCT/US2019/062958

§ 371 Date: May 28, 2021

Related U.S. Application Data
(60) Provisional application No. 62/773,457, filed on Nov. 30, 2018.
(60) Provisional application No. 62/795,218, filed on Jan. 22, 2019.

(51) Int. Cl. G01N 33/574

(52) U.S. Cl. CPC G01N 33/57415

(57) ABSTRACT

Methods and compositions are provided for detecting and/or determining a patient's risk of cancer. The methods utilize categorizing the source of biopsy samples based on patient ancestry mapping in combination with cancer markers to identify patients at higher risk of cancer.

### CROSS-REFERENCE TO RELATED APPLICATIONS

This application claims priority to U.S. Provisional Patent Application Nos. 62/773,457, filed Nov. 30, 2018, and 62/795,218, filed Jan. 22, 2019, the contents of each are incorporated by reference in their entirety into the present application.

### STATEMENT OF GOVERNMENT SUPPORT

This invention was made with government support under W81XWH-15-1-0707 awarded by U.S. Army Medical Research & Material Command. The government has certain rights in the invention.

### BACKGROUND OF THE DISCLOSURE

Recent data demonstrating a correlation between lymph node positivity at the time of detection, and the probability of disease recurrence even decades post detection, only solidifies the principle that the detection of breast cancer prior to lymph node metastasis can appreciably improve clinical outcomes.

Although the last decade witnessed significant improvements in imaging technologies including 3D-mammography, false negatives remain a significant concern. One way to overcome these false negatives is to complement radiologic techniques with molecular assays that measure the “transcriptomic and epigenetic field effect” or the “cancer induced field effect” of tumors on “normal” adjacent tissues (NATs). Teschendorff et al (Nature Communications (2016) 7:10478) demonstrated tumor-induced epigenetic field defects in the NATs and specifically, the targeting of transcription factor binding sites specifying chromatin architecture and stem cell differentiation pathways including Wnt and FGF signaling networks. Unfortunately, the Tumor Genome Atlas (TCGA) of breast cancer utilized reduction mammoplasty or NATs as their controls in transcriptome analyses, which are often used as “normal” controls for comparative analyses of tumors. This limitation was highlighted in another study, which compared TCGA “normal” breast transcriptome with the transcriptome of epithelial cells from normal breast tissues from healthy women. Significant differences were noted between these two sources of normal tissues. Reduction mammoplasty samples are also histologically abnormal compared to breast tissues from healthy women.

While molecular markers of cancers, particularly gene expression signatures, were traditionally developed by comparing gene expression between healthy and cancer tissues, the effects of genetic ancestry on tumor evolution and gene expression are just beginning to be recognized. This observation is highly relevant in the context of known differences in cancer incidence and outcome based on genetic ancestry. For example, women of African Ancestry (AA) suffer higher mortality from the aggressive breast cancer subtype, triple negative breast cancer (TNBC), than women of European Ancestry (EA). Furthermore, basal-like and mesenchymal stem-like TNBCs are significantly more common in AA women. By contrast, breast cancer in Hispanic and Native American women is less prevalent and these women have better outcomes. Whether the worse outcome in AA women is due to an increased incidence of TNBC or unique biological factors that promote aggressive biology is an important but unresolved challenge in cancer disparities research.

Dietze et al. (Nat Rev Cancer (2015) 5(4):248-54) recently highlighted that key molecular pathways, including Aurora A-PLK, EZH2, and Wnt-stem cell signaling networks, are significantly up-regulated in TNBCs of AA women compared to TNBCs of women of EA. Dietze et al. further emphasized that it remains unknown whether genomic aberrations unique to AA women TNBCs result in activation of these signaling pathways or whether basal activity of these pathways in normal breast tissue of AA women is inherently different compared to EA women breast tissue. It remains possible that normal breast tissue biology varies based on genetic ancestry. Evidence for this possibility comes from the recent discovery of breast cancer protective alleles in Latinas. Single nucleotide polymorphisms (SNPs) in the protective allele are located on gene regulatory regions affecting the expression of genes linked to differentiation. Studies have discovered enrichment of a unique population of cells in the normal breast tissue of AA women (Nakshatri et al., Scientific Reports (2015) 5:13526). Furthermore, a breast cancer susceptibility loci in AA women, potentially altering the expression levels of microRNA miR-3065, has recently been discovered (Bensen et al., Breast Cancer Res (2018) 20(1):45).

Breast cancer diagnosis prior to lymph node metastasis can appreciably improve clinical outcomes. While radiologic techniques have significantly improved early diagnosis, molecular markers that can complement radiologic techniques are needed to reduce false positives or negatives. A need also exists to develop tests specific to the genetic ancestry of the patient.

### SUMMARY

The present disclosure is directed to methods and compositions for detecting and/or determining a patient's risk of cancer. The methods utilize categorizing the source of biopsy samples based on patient ancestry mapping, in combination with cancer markers to identify patients at higher risk of cancer. More particularly, the present disclosure shows specific upregulation of zinc-finger E-box-binding homeobox 1 (“ZEB1”)+ cells in cancerous breast tissue in European Ancestry (EA) women but not in African Ancestry (AA) women. Accordingly, an increase in the number of ZEB1+ cells in the breast tissue can be an indicator of the presence of breast cancer in EA women. Breast tissue of AA women naturally contains higher numbers of ZEB1+ cells, which may contribute to the aggressive behavior of AA women's breast cancer. In addition, cancerous breast tissue in AA women shows reduced levels of forkhead box A1 (“FOXA1”)+ cells. Accordingly, staining for the presence of ZEB1+ and FOXA1+ cells by immunohistochemistry coupled with ancestry mapping allow in conjunction with radiological techniques can provide for an improved method of early detection of breast cancer in EA and AA women.

In accordance with one embodiment breast biopsies from the affected breast as well as from contralateral breast are assessed for the presence of ZEB1+ and FOXA1+ cells. DNA from the patient can be analyzed for ancestry markers using commercially available high-discriminatory 41-single nucleotide ancestry markers to ensure that self-reported ethnicity matches at the genomic level.

As disclosed herein both genetic ancestry and the use of breast tissue from clinically healthy women as controls influence the detection of cancer-induced changes in the breast tissue. Applicant shows that alterations in ZEB1+ cells in normal adjacent tissue (NATs) to tumors are observed predominantly in EA women, whereas FOXA1+ cells were altered in the NATs of AA women Immune cell activation in the tumors, as well as, surrounding tissue show genetic ancestry dependent variations as evident from differences in PD1+ and PDL1+ cells in the NATs of AA women and EA women. Thus, biomarker discovery needs to consider not only sample size and statistical methods, but also genetic ancestry and relevant true normal control tissues.

In some embodiments, a method of reducing the number of cancer false positives detected in patients by screening an initial set of potential cancer or pre-cancer biopsy samples to identify samples associated with higher risk of cancer is provided. The method comprises the steps of: obtaining a set of potential cancer or pre-cancer biopsy samples recovered from patient tissues identified by radiology techniques; categorizing the source of said biopsy samples based on patient ancestry mapping; analyzing a breast tissue biopsy sample to determine the numbers of ZEB1+ cells and FOXA1+ cells; identifying patients of European Ancestry having upregulation of ZEB1+ cells relative to true normal breast tissue; identifying patients of African Ancestry reduced levels of FOXA1+ cells relative to true normal breast tissue, wherein said identified patient of European Ancestry and women of African Ancestry are assessed with a higher risk of cancer or higher risk of developing cancer than patients of European Ancestry having normal/average levels of ZEB1+ cells and patients of African Ancestry having normal/average levels of FOXA1+ cells.

In some embodiments, a method of analyzing potential cancer or pre-cancer biopsy samples of patients of European Ancestry or African Ancestry is provided. The method comprises obtaining a set of potential cancer or pre-cancer biopsy samples recovered from patient tissues identified by radiology techniques; categorizing said sample based on ancestry mapping of the patient source of said sample; and quantifying the number of cell that comprise a cancer associated biomarker selected from a group consisting of ZEB1, FOXA1, and GATA Binding Factor 3 (“GATA3”) or a combination thereof. In one embodiment the method comprises quantifying the number of ZEB1+ cells and FOXA1+ cells in a tissue biopsy sample identified as potentially being cancerous by radiological techniques. In some embodiments, the method comprises obtaining a set of potential cancer or pre-cancer biopsy samples recovered from patient tissue identified by radiology techniques; quantifying the expression level of Proto-Oncogene C-Met (“c-MET”) in the biopsy sample, wherein the patient is duffy-null or duffy-heterozygous.

In some embodiments, an improved method of treating cancer in a patient of European Ancestry or African Ancestry is provided. The method comprises the steps of: obtaining a tissue biopsy sample identified as potentially being cancerous or pre-cancerous by radiological techniques and categorized as being from a patient of European Ancestry or African Ancestry; analyzing said sample for upregulation of ZEB1+ cells and reduced levels of FOXA1+ cells compared to a relevant true normal sample or relevant true normal population data; identifying patients of European Ancestry and having specific upregulation of ZEB1+ cells; identifying patients of African Ancestry and having reduced levels of FOXA1+ cells; and treating said identified patients of European Ancestry and patients of African Ancestry using anti-cancer therapies.

In some embodiments, an improved method of analyzing a pre-cancer or cancerous biopsy in a patient who is duffy-null is provided. The method comprises the steps of: analyzing a tissue biopsy sample obtained from said patient and identified as cancerous or pre-cancerous using radiology techniques; and quantifying the levels of phosphorylated c-MET in the patient sample.

In some embodiments, an improved method of analyzing a pre-cancerous or cancerous biopsy sample from a patient of European Ancestry is provided. The method comprises the steps of: analyzing the biopsy sample identified as pre-cancerous or cancerous using radiology techniques; quantifying the levels of GATA3+ cells; and comparing the level of GATA3+ cells to a relevant true normal breast tissue sample or a relevant true normal population data.

In some embodiments, a kit for detecting and quantitating the relative number of ZEB1+ cells and FOXA1+ cells in a tissue sample recovered from a patient is provided. The kit comprises a reagent for the detection of ZEB1+ cells; and a reagent for the detection of FOXA1+ cells. In one embodiment the reagent is an antibody specific for the respective ZEB1 and FOXA1 markers. In one embodiment, the kit further comprises reagents for the detection of GATA3+ cells, optionally wherein the reagent is an antibody specific for the GATA3 marker.

In accordance with one embodiment an improved method of treating cancer in a patient is provided. The method comprises the steps of categorizing said patient based on ancestry mapping and analyzing a tissue biopsy sample recovered from the patient for upregulation of ZEB1+ cells and reduced levels of FOXA1+ cells relative to normal breast tissue. Patients that are identified as being of European Ancestry and having specific upregulation of ZEB1+ cells as well as patients identified as being of African Ancestry and having reduced levels of FOXA1+ cells are then provided with anti-cancer therapies known to those skilled in the art. Such therapies include radiation and/or chemotherapy. In one embodiment the tissue biopsy sample is recovered from human female breast tissue.

### BRIEF DESCRIPTION OF THE DRAWINGS

FIG. 1A-1D: shows ZEB1 expression pattern in KTB-normal, normal adjacent tissues (NATs), and breast tumors. FIG. 1A) shows a representative immunohistochemistry in KTB normal, NATs, and tumors of women of African Ancestry (AA) and European Ancestry (EA). FIG. 1B) are graphs presenting data that demonstrates the differences in ZEB1 expression (positivity and H-score) between KTB-normal of AA and EA women. FIG. 1C) are graphs presenting data that demonstrates the differences between KTB-normal and NATs in AA and EA women. FIG. 1D) are graphs presenting data that demonstrates the differences between NATs and tumors in AA and EA women.

FIG. 2A-2D: shows FOXA1 expression pattern in KTB-normal, normal adjacent to tumor (NATs) and in breast tumors. FIG. 2A) shows representative immunohistochemistry in KTB normal, NATs, and tumors of AA and EA women. FIG. 2B) are graphs presenting data that demonstrates the differences in FOXA1 expression (positivity and H-score) between KTB-normal of AA and EA women. FIG. 2C) are graphs presenting data that demonstrates the differences between KTB-normal and NATs in AA and EA women. FIG. 2D) are graphs presenting data that demonstrates the differences between NATs and tumors in AA and EA women.

FIG. 3A-3D: shows ERα expression pattern in KTB-normal, normal adjacent to tumor (NATs) and in breast tumors. FIG. 3A) shows representative immunohistochemistry in KTB normal, NATs, and tumors of AA and EA women. FIG. 3B) are graphs presenting data that demonstrates the differences in ERα expression (positivity and H-score) between KTB-normal of AA and EA women. FIG. 3C) are graphs presenting data that demonstrates the differences between KTB-normal and NATs in AA and EA women. FIG. 3D) shows differences between NATs and tumors in AA and EA women.

FIG. 4A-4B: are graphs presenting data that present the statistical analyses of (FIG. 4A) CD8 and (FIG. 4B) CD68 positivity in NATs and tumors (T) of AA and EA women. All statistically significant differences are indicated with p values.

FIG. 5A-5B: are graphs presenting data that presents the statistical analyses of (FIG. 5A) PD1 and (FIG. 5B) PDL1 TPS scores in NATs and tumors (T) of AA and EA women. All statistically significant differences are indicated with p values.

FIG. 6 are bar graphs showing the ancestry marker distribution patterns of African American and Caucasian women who donated their breast tissue to KTB and were part of the TMA.

FIG. 7A-7D shows the distribution of MSRB3 within normal, NATs, and tumor cells of AA women and EA women. FIG. 7A shows MSRB3 immunohistochemistry of normal tissue, NATs, and tumors of AA and EA women; FIG. 7B is a graph showing the difference in MSRB3 positivity between normal samples from AA and EA women; FIG. 7C provides two graphs showing the differences between MSRB3 expression in normal tissue and NATs in AA and EA women; and FIG. 7D shows two graphs that show the differences between GATA3 expression in NATs and tumors of AA and EA women.

FIG. 8A-8C are graphs presenting data regarding the expression levels of GATA3; wherein FIG. 8A shows the differences in GATA3 expression between normal tissue of AA and EA women; FIG. 8B shows differences between GATA3 expression in normal tissue and NATs of AA and EA women; and FIG. 8C shows the differences between GATA3 expression in NATs and tumors in AA and EA women.

FIG. 9 presents dot blot data demonstrating duffy-null epithelial cells display higher levels of tyrosine phosphorylated c-MET, IGFR1, and Insulin receptor.

FIG. 10 are photographs of expression data demonstrating duffy-null and duffy-heterozygous epithelial cells contain increased levels of phospho-c-MET and phosphor-Tyr307 GAB 1.

### DETAILED DESCRIPTION

### Abbreviations

The following abbreviations are used throughout the specification:

NATs: “normal” adjacent tissues;

TCGA: Tumor Genome Atlas;

AA: African Ancestry;

EA: European Ancestry;

TNBC: triple negative breast cancer;

TMA: tissue microarray.

### Definitions

In describing and claiming the invention, the following terminology will be used in accordance with the definitions set forth below.

As used herein, the term “treating” includes prophylaxis of the specific disorder or condition, or alleviation of the symptoms associated with a specific disorder or condition and/or preventing or eliminating said symptoms.

As used herein the term “patient” without further designation is intended to encompass any warm blooded vertebrate domesticated animal (including for example, but not limited to livestock, horses, mice, cats, dogs and other pets) and humans.

As used herein, the term “cancer induced field effect” refers to the effect cancer cells have on surrounding phenotypically normal appearing tissues.

As used herein, the term “GATA3” represents GATA Binding Protein 3 or GATA Binding Factor 3. This protein belongs to a family of GATA transcription factors. A GATA3+ cell refers to a cell that expresses GATA3.

As used herein, the term “ERα” is an abbreviation for estrogen receptor alpha.

As used herein the term “ZEB1” is an abbreviation for zinc-finger E-box-binding homeobox 1. A ZEB1+ cell refers to a cell that expresses ZEB1.

As used herein the term “FOXA1” is an abbreviation for forkhead box A1 and is a transcriptional activator protein. FOXA1+ cells refer to cells that express FOXA1.

As used herein the term “c-MET” is an abbreviation for Tyrosine-Protein Kinase Met or Proto-Oncogene C-Met.

As used herein the term “NATs” defines normal adjacent tissue that is located near or next to a cancerous cell or tumor. A “matching NATs or matching NAT” refers to a normal adjacent tissue(s) and the tumor or cancerous cell coming from the same patient.

As used herein the term “Ancestry Mapping” defines a methodology for conducting a molecular analysis of nuclear and organelle genomic nucleic acid sequences recovered from an individual to determine the geographic origin of the ancestors of that individual.

As used herein the term “African Ancestry” is used to define an individual identified based on Ancestry Mapping as having ancestors having a geographic origin of Africa as described in Nievergelt C M et al., Investigative Genetics 2013, 4:13, the contents of which are incorporated herein by reference in its entirety. African Ancestry excludes a combination of genetic markers that would identify a person as predominately Latin Ancestry, Native American Ancestry, Indigent Ancestry, Asian Ancestry, or European Ancestry.

As used herein the term “European Ancestry” is used to define an individual identified based on Ancestry Mapping as having ancestors having a geographic origin of Europe as described in Nievergelt C M et al., Investigative Genetics 2013, 4:13. European Ancestry excludes a combination of genetic markers that would identify a person as predominately Latin Ancestry, Native American Ancestry, Indigent Ancestry, Asian Ancestry, or African Ancestry.

As used herein the term “duffy-null” defines a genetic mutation where the person is lacking expression of protein from both copies of the duffy gene. This mutation was originally discovered in a subset of people with Sub-Saharan African Ancestry.

As used herein the term “duffy-heterozygous” defines a genetic mutation where the person is lacking expression of protein from one copy of the duffy gene.

As used herein the term “TPS” describes a tumor proportion score, which is the percentage of viable tumor cells showing partial or complete membrane staining.

As used herein the term “true normal breast tissue sample” defines a breast tissue sample free of cancer or pre-cancer cells and free of cancer induced field effects. Further the sample is from another healthy individual of at least the same species, gender, ancestry, and age. Additional criteria may be used to narrow down the scope of a true normal breast tissue sample including whether both the sample and the healthy individual were ever pregnant, breast-fed, or undergone any kind of mammoplasty as an example.

As used herein the term “true normal population data” defines an averaged data generated from samples recovered from numerous healthy individuals, wherein each sample obtained from said individuals is a true normal breast tissue sample. Further, the numerous healthy individuals would all be at least the same species, gender, ancestry, and age as the patient sample. Additional criteria may be used to narrow down the scope of a true normal population data including whether both the sample and the numerous individuals were ever pregnant, breast-fed, or undergone any kind of mammoplasty as an example.

As used herein the term “biopsy” defines any solid tissue sample recovered form a patient.

As used herein the term “increased levels” in reference to a biological marker, encompasses increased numbers of cells expressing a biomarker and/or increased total expression of a biomarker. As an illustrative example, increased levels include increased numbers of cells expressing ZEB1 and/or increased total expression of ZEB1 compared to the true normal sample or true normal population data.

As used here the term “decreased levels” in reference to a biological marker, encompasses a decrease in the number of cells expressing a biomarker and/or a decrease in the total expression of a biomarker. As an illustrative example, decreased levels include decreased numbers of cells expressing FOXA1 or GATA3 and/or decreased total expression of FOXA1 or GATA3 compared to the true normal sample or true normal population data.

### EMBODIMENTS

In one embodiment the present disclosure is directed to an improved method for detecting cancer and reducing the number of false positives associated with traditional radiological techniques for detecting cancer.

The method combines quantitative assessment of cancer associated markers with ancestry information to more accurately assess the impact of altered expression levels relative to normal tissues recovered from healthy individuals having the same or similar ancestry. In one embodiment the method is used to enhance the accuracy of known techniques for detecting solid tumors. Expression levels of the relevant cancer associated markers can be quantitatively measured by methods known by those skilled in the art such as, for example, immunohistochemistry, immunofluorescence, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, enzyme linked immunosorbent assay (ELISA), in situ hybridization, and combinations thereof.

In some embodiments, the pre-cancer or cancer is breast cancer. In some embodiments, the pre-cancer or cancer is triple negative breast cancer, wherein the cancer cells test negative for estrogen receptors, progesterone receptors, and HER2 protein. In some embodiments, the pre-cancer or cancer is HER2+ breast cancer. In some embodiments, the pre-cancer or cancer is HER2− breast cancer. In some embodiments, the pre-cancer or cancerous cells are EpCAM In some embodiments, the pre-cancer or cancerous cells are ERα+. In some embodiments, the pre-cancer or cancerous cells are ERα−.

In some embodiments, the patient is male or female. In some embodiments, the patient is a female. In some embodiments, the patient is a woman.

In some embodiments, the method comprises detecting the presence, and optionally the relative concentration, of a cancer associated biomarker. In some embodiments, the cancer associated biomarker or “biomarker” is selected from a group consisting of ZEB1, GATA3, FOXA1, c-MET, ERα, PDLI, PD1, CD68, and CD8.

In one embodiment a method of analyzing potential cancer or pre-cancer biopsy samples of patients of European Ancestry or African Ancestry is provided.

The method comprises obtaining a set of potential cancer or pre-cancer biopsy samples recovered from patient tissues identified by radiology techniques or other screening techniques; categorizing said sample based on ancestry mapping of the patient source of said sample; and quantifying the number of cells comprising a cancer associated biomarker selected from a group consisting of ZEB1, FOXA1, and GATA3, or a combination thereof. In one embodiment, the final step of the method comprises quantifying

i) the number of cells comprising the biomarker ZEB1; and the number of cells comprising the biomarker FOXA1; or

ii) the number of cells comprising the biomarker ZEB1; and the number of cells comprising the biomarker GATA3; or

iii) the number of cells comprising the biomarker FOXA1 and the number of cells comprising the biomarker GATA3; or

iv) the number of cells comprising the biomarker ZEB1; the number of cells comprising the biomarker FOXA1; and the number of cells comprising the biomarker GATA3. In one embodiment the method comprises quantifying the number of cells comprising the biomarker ZEB1; and the number of cells comprising the biomarker FOXA1.

In some embodiments, a method of reducing the number of cancer false positives detected in patients by screening an initial set of potential cancer or pre-cancer biopsy samples to identify samples associated with higher risk of cancer is provided. The method comprises the steps of: obtaining a potential cancer or pre-cancer biopsy sample from patient tissues identified as potentially cancerous or pre-cancerous by radiology techniques or use of associated cancer markers; categorizing the source of said biopsy samples based on patient ancestry mapping; analyzing the tissue biopsy sample to determine the numbers of ZEB1+ cells and FOXA1+ cells; identifying patients of European Ancestry having upregulation of ZEB1+ cells relative to true normal breast tissue; identifying patients of African Ancestry having reduced levels of FOXA1+ cells relative to true normal breast tissue, wherein said identified patient of European Ancestry and women of African Ancestry are assessed with a higher risk of cancer or higher risk of developing cancer than patients of European Ancestry having normal levels of ZEB1+ cells and patients of African Ancestry having normal levels of FOXA1+ cells. As used herein “normal” is defined as the range of values found in comparable healthy tissues of individuals free of disease. In one embodiment the normal range for a given test is based on the results that are seen in 95% of the healthy population. Identified individuals are those that have detected levels of the relevant marker outside the normal range of comparable (i.e. ancestry matched) sourced tissues. In one embodiment patients of European Ancestry having upregulation of ZEB1+ cells by more than 1%, 2%, 5%, 10% or more from the highest normal value for true normal breast tissue; and patients of African Ancestry having reduced levels of FOXA1+ cells by more than 1%, 2%, 5%, 10% or more from the lowest normal value for true normal breast tissue are identified as the identified individuals having an higher risk of having or developing cancer. In some embodiments, the tissue biopsy sample is recovered from breast tissue. In some embodiments, the patient is a woman.

In some embodiments, a method of analyzing potential cancer or pre-cancer biopsy samples of patients of European Ancestry or African Ancestry is provided. The method comprises obtaining a set of potential cancer or pre-cancer biopsy samples recovered from patient tissues identified by radiology techniques; categorizing said sample based on ancestry mapping of the patient source of said sample; and quantifying the number of cells expressing a cancer associated biomarker selected from a group consisting of ZEB1, FOXA1, GATA3, and phospho-c-MET or a combination thereof.

In some embodiments the method further comprises comparing the number of cells expressing the biomarker in said sample with a relevant true normal sample or relevant true normal population data. In some embodiments, the biomarker is ZEB1. In some embodiments, the biomarker is FOXA1. In some embodiments, the biomarker is GATA3. In some embodiments, the biomarker is phospho-c-MET. In some embodiments the biomarker is ZEB1 and FOXA1. In some embodiments, the biomarker is selected from a group consisting of ZEB1, FOXA1, GATA3, and phospho-c-MET, or a combination thereof, and further includes at least one other PDL1, PD1, CD68, CD8, ERα, or a combination thereof.

In some embodiments, the relevant true normal sample is provided by a woman of African Ancestry. In some embodiments, a relevant true normal population data comprising samples from African Ancestry (AA) women is used.

In some embodiments, the relevant true normal sample is provided by a woman of European Ancestry. In some embodiments, a relevant true normal population data comprising samples from European Ancestry (EA) women is used.

In some embodiments, the method further comprises screening said biopsy sample for duffy-null or duffy-heterozygous alleles. In some embodiments, the method further comprises screening said biopsy sample for duffy-null alleles. In some embodiments, the method further comprises quantifying the expression level of phospho-c-MET in said biopsy sample. In some embodiments, the expression level of phospho-c-MET is compared to a matching NATs or a relevant true normal sample or relevant true normal population data.

In some embodiments, the biopsy sample is recovered from human female breast tissue.

In some embodiments, the biopsy sample is categorized as being either European Ancestry or African Ancestry.

In some embodiments, the number of ZEB1+ cells and FOXA1+ cells are compared to a relevant true normal sample or a relevant true normal population data. In some embodiments, the number of GATA3+ cells is compared to a relevant true normal sample or a relevant true normal population data. In some embodiments, the level of phospho-c-MET expression in the sample biopsy is compared with a relevant true normal sample or a relevant true normal population data.

In some embodiments, the method further comprises, comparing the number of ZEB1+ cells and FOXA1+ cells to a matching normal adjacent tissues (NATs).

In some embodiments, an improved method of treating cancer in a patient of European Ancestry or African Ancestry is provided. The method comprises the steps of: obtaining a tissue biopsy sample identified as being cancer by radiological techniques and categorized as being from a patient of European Ancestry or African Ancestry; analyzing said sample for upregulation of ZEB1+ cells and reduced levels of FOXA1+ cells compared to a relevant true normal sample or relevant true normal population data; identifying patients of European Ancestry and having specific upregulation of ZEB1+ cells; identifying patients of African Ancestry and having reduced levels of FOXA1+ cells; and treating said identified patients of European Ancestry and patients of African Ancestry using anti-cancer therapies. In some embodiments, the patient is a woman and the biopsy sample is recovered from breast tissue.

In some embodiments, a patient is identified as having, or at risk of developing, cancer, when a patient of European Ancestry has an increase of 1, 5, 10, 15, 20, 25, or 50% in the numbers of ZEB1+ cells relative to true normal breast tissue and when a patient of African Ancestry has a reduction of 1, 5, 10, 15, 20, 25, or 50% of the number of FOXA1+ cells relative to true normal breast tissue. In some embodiments, the ZEB1+ cells or FOXA1+ cells comprise the patient's normal adjacent tissue (NAT).

In some embodiments, an improved method of analyzing a pre-cancer or cancerous biopsy in a patient who is duffy-null is provided. The method comprises the steps of: analyzing a tissue biopsy sample obtained from said patient and identified as cancerous or pre-cancerous using radiology techniques; and quantifying the levels of phospho-c-MET in the patient sample. In some embodiments, the method further comprises quantifying the levels of ZEB1+ cells in said biopsy sample. In some embodiments, the method further comprises comparing the levels of phospho-c-MET to a relevant true normal sample or a relevant true normal population data.

In some embodiments, a kit for detecting and quantitating the relative number of ZEB1+ cells and FOXA1+ cells in a tissue sample recovered from a patient is provided. In one embodiment the kit comprises a reagent for the detection of ZEB1+ cells; and a reagent for the detection of FOXA1+ cells. In some embodiments, the reagent for detecting ZEB1+ cells is an antibody specific for ZEB1. In some embodiments, the reagent for detecting FOXA1+ cells is an antibody specific for FOXA1.

In one embodiment, the kit comprises reagents for conducting the ancestry mapping as well as reagents for detecting and quantitating the presence of ZEB1+ cells and FOXA1+ cells in a tissue sample recovered from a patient. In accordance with one embodiment, a kit for detecting and quantitating the relative number of ZEB1+ cells and FOXA1+ cells in a tissue sample recovered from a patient comprises a reagent for the detection of ZEB1+ cells, and a reagent for the detection of FOXA1+ cells. In one embodiment, the reagents are antibodies. In one embodiment, the kit comprises one or more of the following antibodies: FOXA1 (Santa Cruz sc-6553), PD1 (Cell Marque 315M-98), PDL1 (Keytruda) (clone 22c3, Dako IHC 22c3) and ZEB1 (3G6, cat no 14-9741-82, eBioscience). The kit may further comprise reagents for conducting ancestry mapping. In some embodiments, the kit further comprises a reagent for detecting GATA3, including for example an antibody specific for detecting GATA3. In one embodiment the kit further comprises a reagent for detecting phospho-c-MET. In some embodiments, the reagent is an antibody for detecting phospho-c-MET. The kit typically further comprises instructional materials.

In some embodiments, an improved method of analyzing a pre-cancerous or cancerous biopsy sample from a patient of European Ancestry is provided. The method comprises the steps of analyzing the biopsy sample identified as pre-cancerous or cancerous using radiology techniques; quantifying the levels of GATA3+ cells; and comparing the level of GATA3+ cells to a relevant true normal breast tissue sample or a relevant true normal population data.

In some embodiments, identifying patients of European Ancestry having a reduction of 1, 5, 10, 15, 20, 25, or 50% of the number of GATA3+ cells is an indication of pre-cancer or cancer.

In an illustrative embodiment, decreased levels of GATA3+ cells is an indication of pre-cancer or cancer. In some embodiments, the method further comprises quantifying the levels of ZEB1+ cells and comparing the ZEB1+ cell level to a relevant true normal breast tissue sample.

Various functions and advantages of these and other embodiments of the present disclosure will be more fully understood from the examples shown below. The examples are intended to illustrate the benefits of the present disclosure, but do not exemplify the full scope of the disclosure.

### Clause List

Clause 1. A method of reducing the number of cancer false positives detected in patients by screening an initial set of potential cancer or pre-cancer biopsy samples to identify samples associated with higher risk of cancer, said method comprising the steps of

obtaining a set of potential cancer or pre-cancer biopsy samples recovered from patient tissues, optionally wherein the potential cancer or pre-cancer biopsy samples were identified by radiology techniques or other cancer detection techniques;

categorizing the source of said biopsy samples based on patient ancestry mapping;

analyzing the tissue biopsy sample to determine the numbers of ZEB1+ cells and FOXA1+ cells;

identifying patients of European Ancestry having upregulation of ZEB1+ cells relative to true normal tissue;

identifying patients of African Ancestry reduced levels of FOXA1+ cells relative to true normal tissue, wherein said identified patient of European Ancestry and women of African Ancestry are assessed with a higher risk of cancer or higher risk of developing cancer than patients of European Ancestry having normal levels of ZEB1+ cells and patients of African Ancestry having normal levels of FOXA1+ cells.

Clause 2. The method of clause 1 wherein a patient is identified as being of African Ancestry or European Ancestry based on analysis using a discriminative 41-ancestry marker profiles (see Nievergelt C M et al., Investigative Genetics 2013, 4:13).
Clause 3. The method of clause 1 or 2 wherein said tissue biopsy sample is recovered from breast tissue and said true normal tissue is true normal breast tissue.
Clause 4. The method of any one of clauses 1-3 wherein the patient is a woman.
Clause 5. A method of analyzing potential cancer or pre-cancer biopsy samples of patients of European Ancestry or African Ancestry, said method comprising:

obtaining a set of potential cancer or pre-cancer biopsy samples recovered from patient tissues identified by radiology techniques or other cancer associated marker;

categorizing said sample based on ancestry mapping of the patient source of said sample; and

quantifying the number of cells expressing ZEB1 and the number of cells expressing FOXA1.

Clause 6. The method of clause 5 further comprising a step of quantifying the number of cells expressing GATA3.
Clause 7. The method of clause 5 or 6 further comprising a step of quantifying the number of cells expressing phospho-c-MET.
Clause 8. A method of analyzing potential cancer or pre-cancer biopsy samples of patients of European Ancestry or African Ancestry, said method comprising:

obtaining a set of potential cancer or pre-cancer biopsy samples recovered from patient tissues identified by radiology techniques or other cancer associated marker;

categorizing said sample based on ancestry mapping of the patient source of said sample; and

quantifying the number of first cells expressing a cancer associated biomarker selected from a group consisting of ZEB1, FOXA1, GATA3, and phospho-c-MET and

quantifying the number of second cells expressing a cancer associated biomarker selected from a group consisting of ZEB1, FOXA1, GATA3, and phospho-c-MET, wherein the first and second cells express different markers selected from the group consisting of ZEB1, FOXA1, GATA3, and phospho-c-MET.

Clause 8.5 The method of clause 8 wherein said first cells express a marker selected from the group consisting of ZEB1, FOXA1, and GATA3 and said second cells express markers selected from the group consisting of ZEB1, FOXA1, and GATA3.
Clause 9. The method of any one of clause 5 to 8 further comprising comparing the biomarker in said sample with a relevant true normal sample or relevant true normal population data.
Clause 10. The method of clause 9, wherein the relevant true normal sample is provided by a woman of African Ancestry.
Clause 11. The method of clause 9, wherein the relevant true normal sample is provided by a woman of European Ancestry.
Clause 12. The method of any one of clause 5 to 11 further comprising screening said biopsy sample for duffy-null alleles.
Clause 13. The method of any one of clause 5 to 12 wherein the biopsy sample is recovered from human female breast tissue.
Clause 14. The method of clause 12 or 13 wherein the biopsy sample is categorized as being either European Ancestry or African Ancestry.
Clause 15. The method of clause 5 wherein the number of ZEB1+ cells and FOXA1+ cells are compared to a relevant true normal sample.
Clause 16. The method of clause 15 further comprising, comparing the number of ZEB1+ cells and FOXA1+ cells to a matching normal adjacent tissues (NATs).
Clause 17. An improved method of treating cancer in a patient of European Ancestry or African Ancestry, said method comprising the steps of

obtaining a tissue biopsy sample identified as being cancer by radiological techniques and categorized as being from a patient of European Ancestry or African Ancestry;

analyzing said sample for increased number of ZEB1+ cells and reduced levels of FOXA1+ cells compared to a relevant true normal sample or relevant true normal population data;

identifying patients of European Ancestry and having specific increase in the number of ZEB1+ cells;

identifying patients of African Ancestry and having reduced number of FOXA1+ cells; and

treating said identified patients of European Ancestry and patients of African Ancestry using anti-cancer therapies.

Clause 18. The method of clause 17 wherein the patient is a woman and the biopsy sample is recovered from breast tissue.
Clause 19. An improved method of analyzing a pre-cancer or cancerous biopsy in a patient who is duffy-null, said method comprising the steps of

analyzing a tissue biopsy sample obtained from said patient and identified as cancerous or pre-cancerous using radiology techniques; and

quantifying the levels of c-MET in the patient sample.

Clause 20. The method of clause 19, further comprising quantifying the levels of ZEB1+ cells in said biopsy sample.
Clause 21. The method of clause 19, further comprising comparing the levels of phospho-c-MET to a relevant true normal sample or a relevant true normal population data.
Clause 22. A kit for detecting and quantitating the relative number of ZEB1+ cells and FOXA1+ cells in a tissue sample recovered from a patient, said kit comprising

a reagent for the detection of ZEB1+ cells; and

a reagent for the detection of FOXA1+ cells.

Clause 23. The kit of clause 22 wherein the reagent is an antibody specific for the respective protein.
Clause 24. The kit clause of clause 22 or 23 further comprising a reagent for detecting GATA3.
Clause 25. The kit clause of any one of clauses 22-24 further comprising a reagent for detecting phospho-c-MET.
Clause 26. The kit clause of 24 and 25, wherein the reagent is an antibody specific for the respective protein.
Clause 27. An improved method of analyzing a pre-cancerous or cancerous biopsy sample from a patient of European Ancestry, said method comprising the steps of

analyzing the biopsy sample identified as pre-cancerous or cancerous using radiology techniques;

quantifying the levels of GATA3+ cells; and

comparing the level of GATA3+ cells to a relevant true normal breast tissue sample or a relevant true normal population data.

Clause 28. The method of clause 27, wherein lowered levels of GATA3+ cells is an indication of pre-cancer or cancer.
Clause 29. The method of clause 27 further comprising quantifying the levels of ZEB1+ cells and comparing the ZEB1+ cell level to a relevant true normal breast tissue sample.
Clause 30. The method of clause 29, wherein an increased level of ZEB1+ cells is an indication of pre-cancer or cancerous breast tissue.
Clause 31. The method of any of the preceding clauses wherein the biopsy comprises NATs.
Clause 32. The method of any of the preceding clauses wherein the biopsy comprises a tumor.

### Example 1

Genetic ancestry influences evolutionary pathways of cancers. For a discussion see Cancer Cell 34:529), the contents of which are incorporated herein by reference. However, whether ancestry influences cancer-induced field effects is unknown. Here the ancestry-mapped relevant true normal breast tissues were utilized as controls to identify cancer-induced field defects in NATs in AA and EA women. As disclosed herein, applicants have taken advantage of genetic ancestry mapped relevant true normal breast tissues to identify differences between relevant true normal and NATs, which can potentially develop into earlier markers of breast cancer initiation. A tissue microarray (TMA) comprising breast tissues from clinically normal breast tissue, NATs, and tumors was analyzed for markers that are expressed in cells with stem and mature luminal cell properties. Applicants also examined the TMA for CD8+ T cells, CD68+ macrophages, PD1+ immune cells and PDL1+ epithelial cells to determine whether immune cell composition of tumors and NATs in AA women differ from that of EA women with the goal of identifying cancer-induced field defects in normal tissue adjacent to breast tumors (NATs) in women of African Ancestry (AA) and European ancestry (EA).

This disclosure will help in early detection of breast cancer. It will also help in resolving ambiguity in breast cancer diagnosis by radiology techniques such as mammography. Biopsy of sites with ambiguity and staining for FOXA1 and ZEB1+ cells provide additional evidence for the presence of cancer. We propose that cancer-induced field defects cause increase in ZEB1+ cells in women of European Ancestry and decrease in FOXA1+ cells in women of African Ancestry.

### Materials and Methods:

Generation of a tissue microarray (TMA). Breast core biopsies from healthy women donated to Komen Tissue Bank (KTB) and surgical material left over after pathologic assessments as part of a treatment protocol were obtained after informed written consent from the subjects. Applicant created a tissue microarray (TMA) comprising: 1) healthy breast tissue from the KTB (KTB-normal), 2) matched NATs, and 3) tumor tissue. About 50 samples from AA women and about 50 samples from EA women were provided for a total of about 300 samples. KTB-normal tissues were age and ancestry mapping matched to NATs/Tumors. BMI of AA women who donated tissues to KTB was 32.3±9, and 28.3±8.5 in case of EA women. Each tumor sample and matching NAT were spotted in duplicate.

### Example 2

Immunohistochemistry and statistical analyses. The TMA was analyzed for ZEB1, methione sulfoxide reductase B3 (“MSRB3”), estrogen receptor alpha (ERα), GATA3, and FOXA1 expression. All immunohistochemistry (IHC) was done in a CLIA-certified histopathology lab and evaluated by three pathologists in a blinded manner and quantitative measurements were done using automated Aperio Imaging system and analysis was done using an FDA approved algorithm. Positivity and H-scores were scored and statistically analyzed as described previously (Breast Cancer Res Treat (2015) 152(3):519-31). With respect to PD1 and PDL1, a tumor proportion score (TPS) was created. The TPS describes the ratio of positive viable tumor cells against all viable tumor cells. The PDL1 TPS followed the prescribed FDA reading: a negative score having <1% positive staining on the cell membrane, a positive score having 1%-49% cells partially or completely expressing PDL1 on the cell membrane, and a strong positive having >50% tumor cells partially or completely expressing PDL1 on the cell membrane at a stronger intensity (Modern Pathol 29:1165-72; NSCLC-Keytruda PDL1 IHC 22C3 pharmDX Interpretation manual). Data were analyzed in three different ways: 1) Expression differences between AA and EA KTB-normal; 2) Expression differences between KTB-normal and NATs; and 3) Expression differences between NATs and tumors. Non-parametric Wilcoxon rank-sum tests were used for unpaired analyses, as positivity and H scores were not normally distributed, whereas non-parametric Wilcoxon signed-rank tests were used for paired analyses. The following antibodies were used: CD8 (Dako IR623), CD68 KP1 (Dako IR609), ER clone:EP1 (Dako IR 084), FOXA1 (Santa Cruz sc-6553), GATA3 (Santa Cruz sc-268), MSRB3 (HPA014432, rabbit polyclonal, Sigma), PD1 (Cell Marque 315M-98), PDL1 (Keytruda) (clone 22c3, Dako IHC 22c3) and ZEB1 (3G6, cat no 14-9741-82, eBioscience).

### Example 3

ZEB1+ cells are enriched in the normal breast tissue of AA compared to EA women. In the mouse mammary gland, PROCR+/EpCAM− cells are suggested to function as multi-potent stem cells. In a previous study focused on evaluating ethnicity-dependent differences in the normal breast tissue, specific enrichment of PROCR+/EpCAM− in cultured normal breast epithelial cells from biopsies of healthy AA women compared to EA women was observed (Nakshatri et al., Scientific Reports (2015) 5:13526). These cells are enriched for the expression of stemness-related transcription factor ZEB1 and have enhanced Wnt pathway activity compared to PROCR±/EpCAM+ cells. ZEB1 has recently been demonstrated to limit onco-suppressive p53-driven DNA damage response in stem cells and thus increase the stem cells' intrinsic susceptibility to malignant transformation. ZEB1+ cells co-express the methionine sulfoxidase reductase (MSRB3), which protects against DNA damage. These observations raised the possibility that PROCR+/ZEB1+ cells are naturally present at higher levels in the normal breast tissue of AA women and failure to consider natural variation in gene expression pattern, influenced at least partially by genetic ancestry, could have an impact on identifying cancer-induced field effect on adjacent normal breast tissue. Measuring PROCR itself in the breast tissue is complicated because there are four haplotypes of PROCR due to SNPs and only one among them is a cell surface protein. Since ZEB1 expression is enriched in PROCR+/EpCAM− cells, applicant used ZEB1 as a surrogate marker for PROCR+/EpCAM− cells in un-manipulated breast tissues.

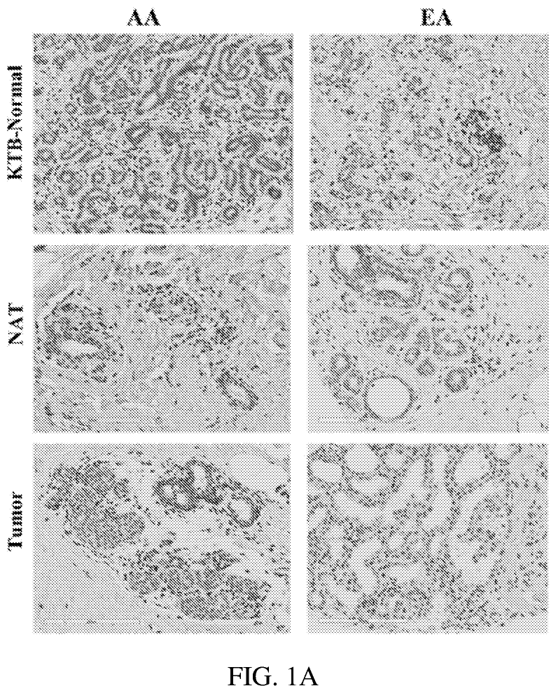

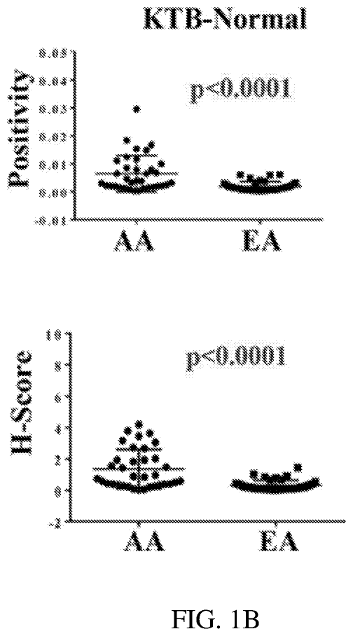

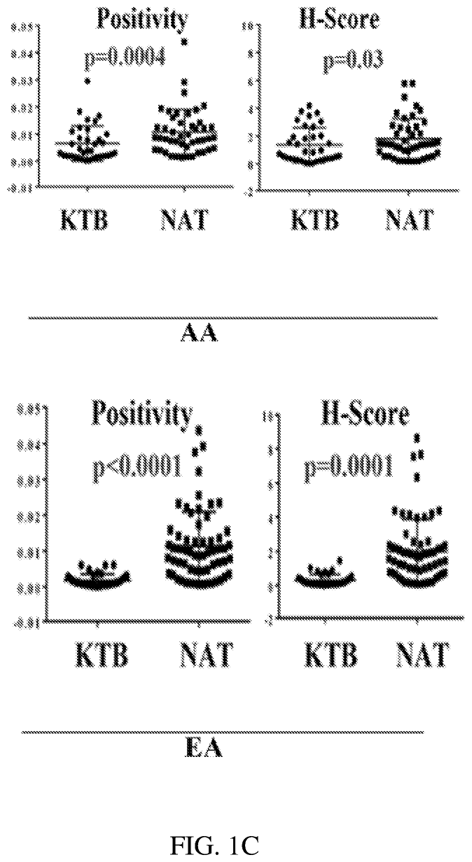

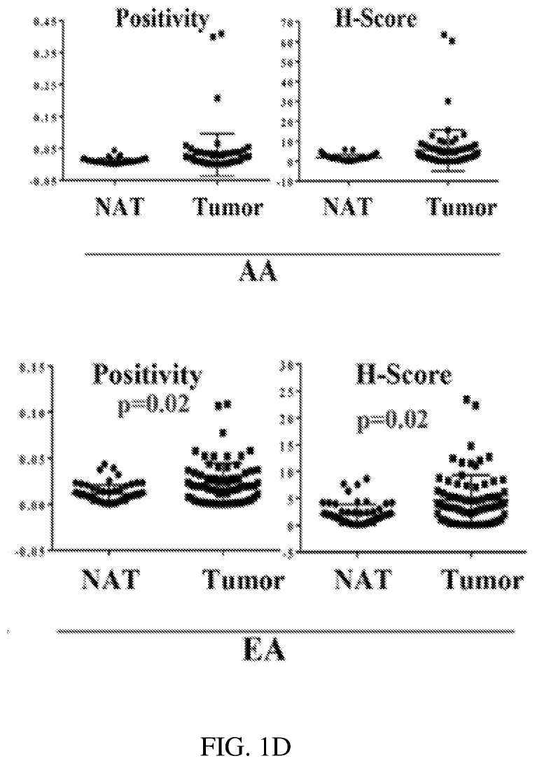

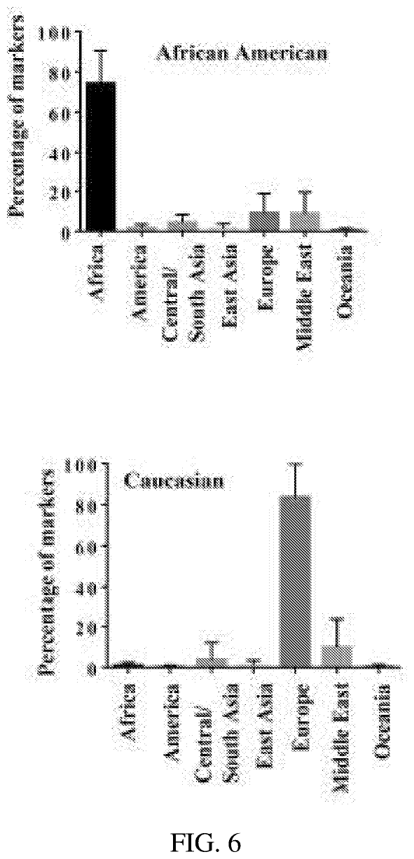

Representative IHC staining patterns of ZEB1 in KTB-normal, NATs, and tumors from AA and EA women were conducted and statistical analyses are presented in FIG. 1B-1D and in Table 1. Descriptive statistics of ethnicity, age, menstrual status, pregnancy, breastfeeding, hormone replacement therapy and family history of breast cancer for the KTB-normal cohort is shown in Table 2. Highly discriminative 41-ancestry marker profiles of KTB-normal showed >75% African ancestry markers in samples from African American women and >80% European ancestry markers in Caucasian women for the generation of a relevant true normal sample or relevant true normal population data (FIG. 6). Characteristics of breast cancer in the tumor cohort are shown in Table 3. ZEB1 expressing cells are localized outside the ductal structures of the normal breast tissue and in the stromal part of the tumors. KTB-normal breast tissue of AA women contained significantly higher levels of ZEB1-positive cells compared to KTB-normal breast tissue of EA women (FIG. 1B). NATs of AA women showed a modest increase in ZEB1+ cells compared to KTB-normal (FIGS. 1C and 1D). The situation was completely different in EA women; both NATs and tumors contained significantly higher levels of ZEB1+ cells compared to KTB-normal (FIGS. 1C and D). NAT to tumor differences were noted only in EA women where an increase in ZEB1+ cells was noted predominantly in ERα+ tumors (Table 4). Thus, ZEB1+ cells are intrinsically higher in the normal breast tissue of AA women, whereas remarkably elevated ZEB1+ cells in the breast tissue of EA women were observed only in the context of breast cancer.

### Example 4

Increase in ZEB1+ cells in KTB-normal of AA women compared to EA women is less likely related to BMI differences, as when analysis was done by subdividing women above and below BMI of 30, irrespective of genetic ancestry, ZEB1 H-score but not positivity showed a marginal relationship (p=0.04) to BMI above and below 30 (Table 5).

### Example 5

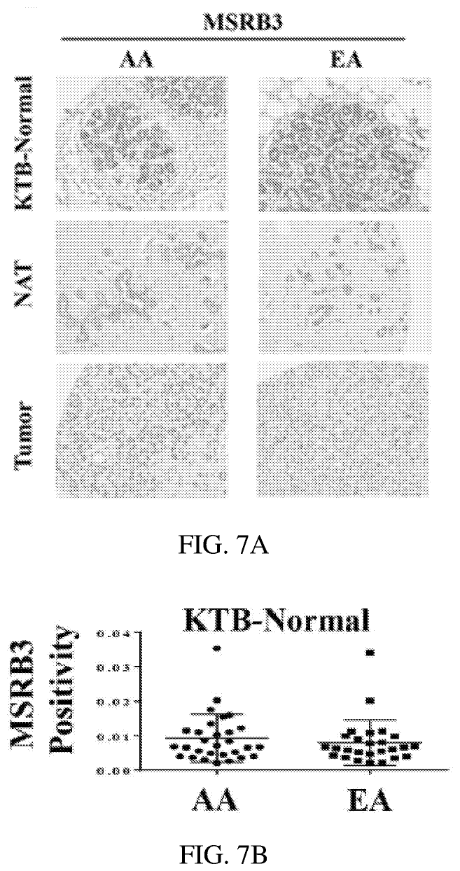

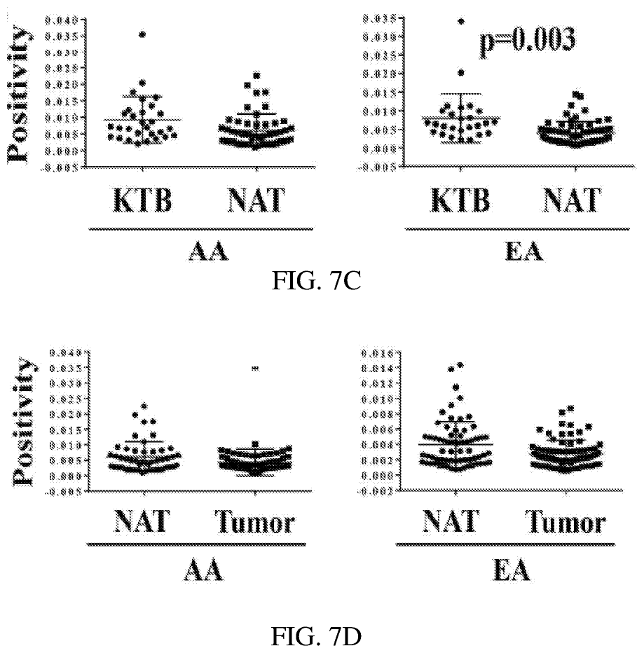

MSRB3 has recently been shown to be one of the downstream transcriptional targets of ZEB1 and it cooperates with ZEB1 during transformation of stem-like cells. To correlate ZEB1 expression with its activity, we measured the levels of MSRB3 using the same antibody used in the above study. We could measure positivity but not H-score because of low-level expression. The expression pattern was similar to that of ZEB1, as cells surrounding the ducts showed expression (FIG. 7A. However, KTB-normal tissues of AA and EA women expressed similar levels of MSRB3 (Table 1 and FIG. 7B, which could be due to regulation by other transcription factors or to the low expression levels, making data interpretation difficult. Furthermore, except for a modest change in expression in NATs compared to KTB normal tissues, no other differences were noted (FIGS. 7C and 7D).

### Example 6

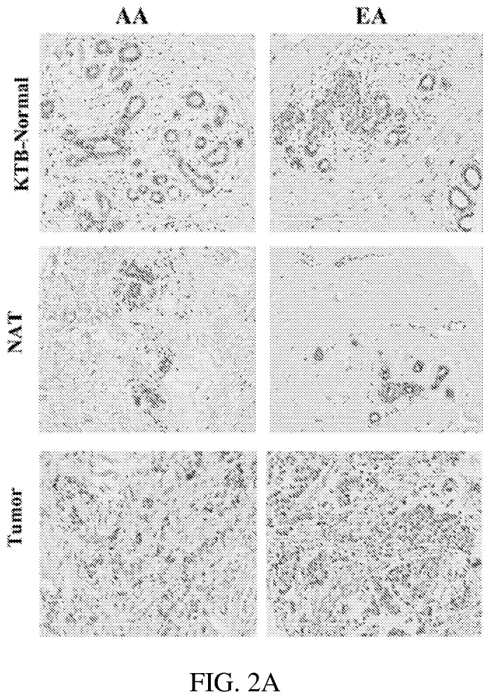

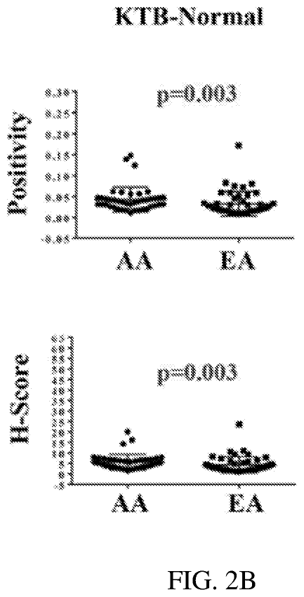

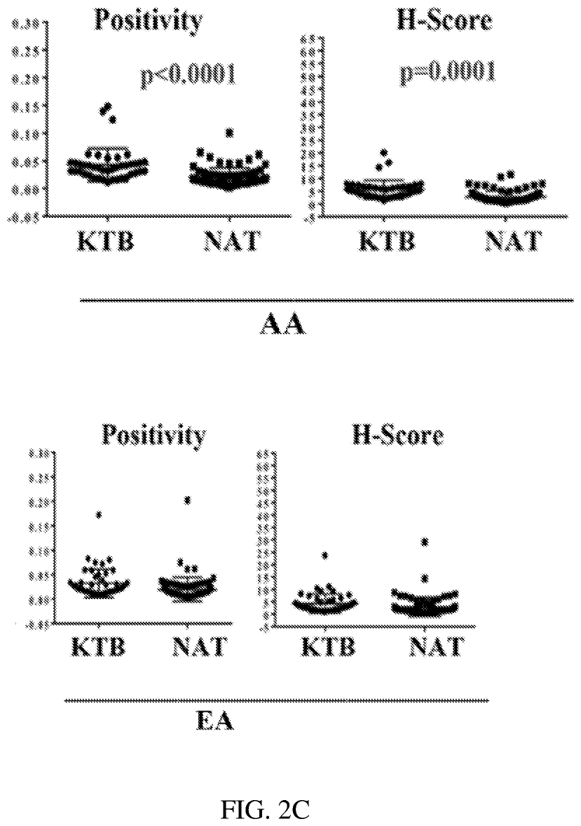

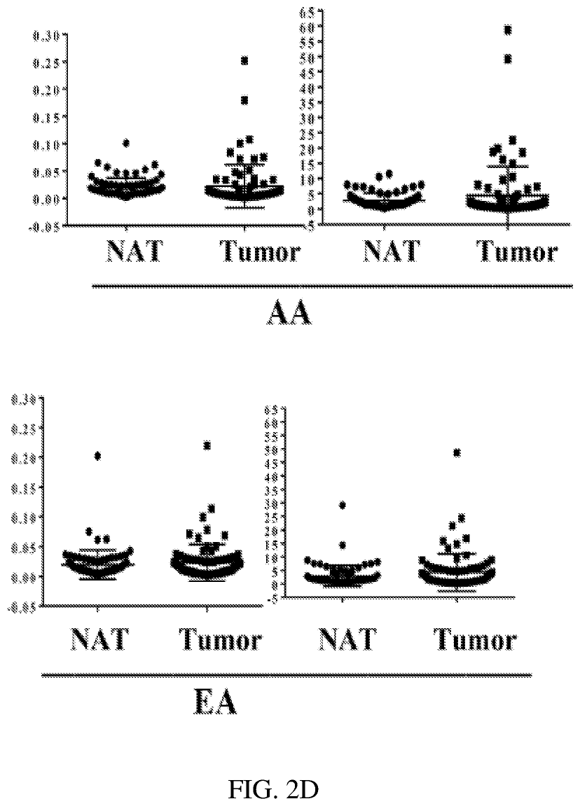

FOXA1 expression is lower in NATs of only AA women: FOXA1 serves as a pioneer factor that controls chromatin access of various nuclear receptors including ERα and controls luminal gene expression. FOXA1 along with another pioneer factor GATA3 and ERα form a lineage restricted hormone-responsive signaling network in the normal breast tissue. While higher expression of FOXA1 in the primary tumor is associated with better outcome, its overexpression in metastatic and/or anti-estrogen resistant tumors is associated with rewiring of ERα signaling and poor outcome. In addition, it is suggested that FOXA1 gene is preferentially methylated in tumors of AA women. Because of its relative importance in breast cancer, we assessed our TMA for FOXA1 expression. Staining patterns of FOXA1 were evaluated and numerical values are presented in Table 1. While FOXA1 levels in KTB-normal of AA women was modestly higher than EA women (FIG. 2B), NATs of AA women had lower FOXA1 compared to KTB-normal. Thus, tumors through their field effect may decrease FOXA1 in the NATs of AA women.

### Example 7

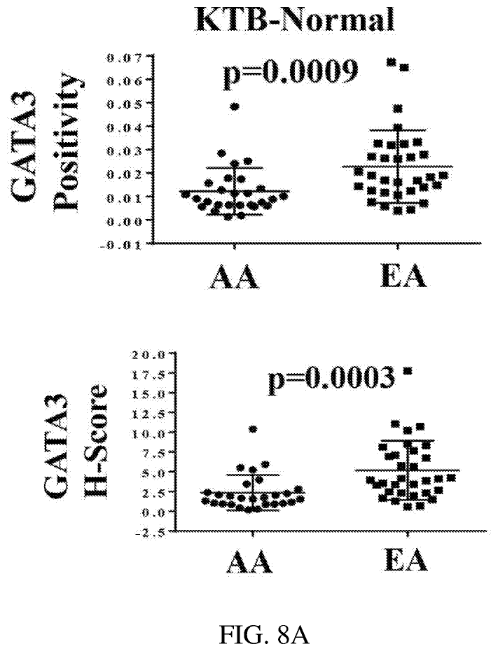

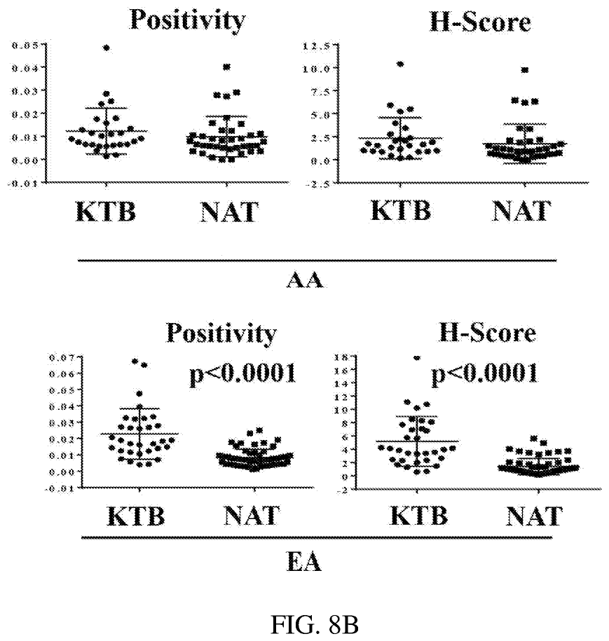

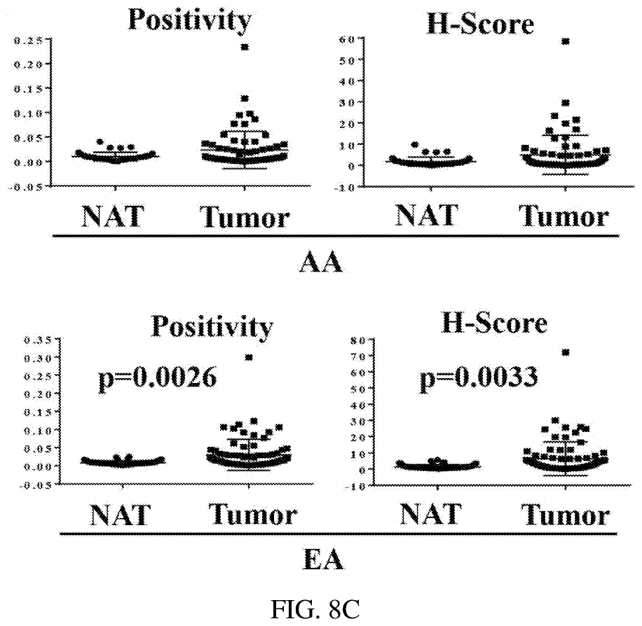

GATA3 levels are higher in KTB-normal of EA compared to AA women: We also examined expression levels of GATA3 to determine whether hormonal signaling networks show genetic ancestry-dependent variation. Consistent with this possibility, GATA3 H-score and positivity were higher in KTB-normal tissues of EA women compared to those of AA women (Table 1 and FIG. 8A. Furthermore, GATA3 is a likely candidate for cancer-induced field effects in EA women as its levels were significantly lower in NATs of EA but not AA women compared to their KTB-normal counterparts (FIG. 8B).

### Example 8

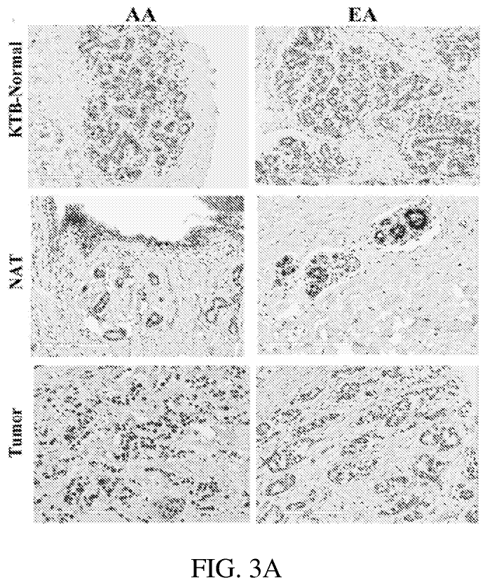

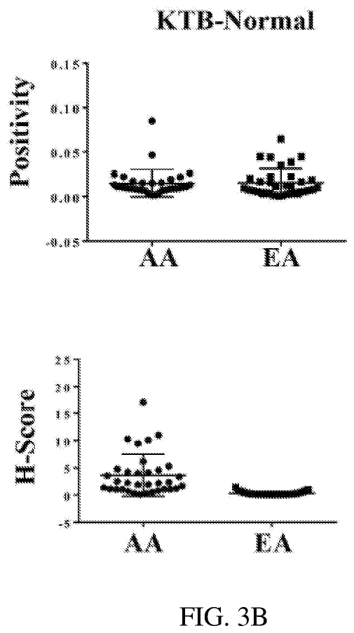

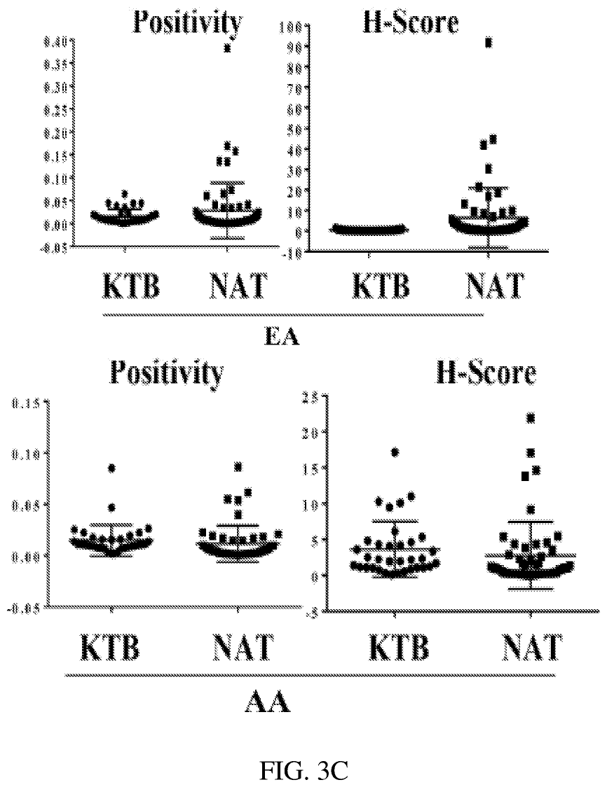

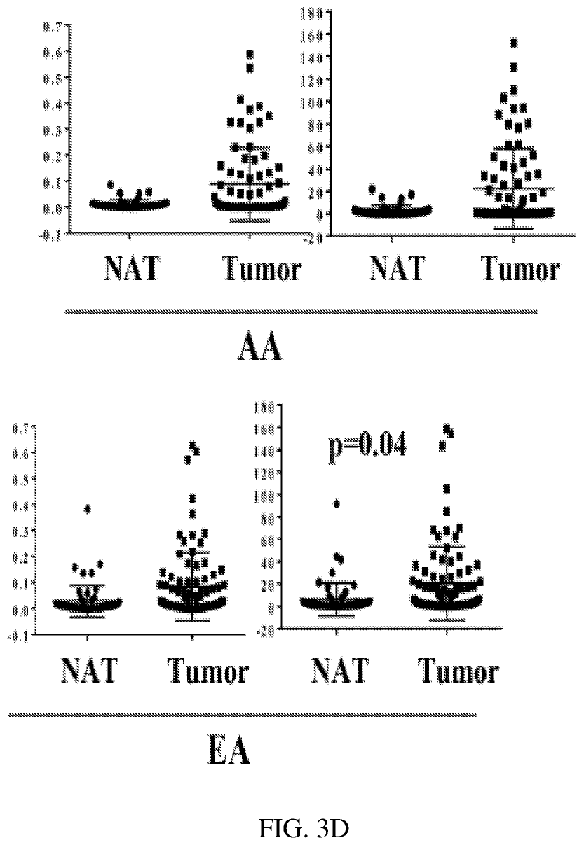

ERα+ cells remain stable: ERα-positive cells in the normal breast tissue are considered to be highly differentiated non-proliferative cells and control proliferation of ERα-negative cells through paracrine mechanisms. Representative ERα staining pattern and statistical analyses are presented in FIG. 3B-3D and Table 1. There were no differences between AA and EA women in ERα-positivity in KTB-normal and NATs. Neither KTB normal tissues nor NATs showed genetic ancestry-dependent differences in ERα levels. The results are not only relevant, but also reassure that the TMA detects only specific changes.

TABLE 1
Differences in expression levels of ER, ZEB1, GATA3, MSRB3, and FOXA1 in KTB normal
between women of African Ancestry and European ancestry wherein the last column shows
the two-sided Wilcoxon Test p-values.

| Variable | African Ancestry True Normal | | | | European Ancestry True Normal | | | | |
|---|---|---|---|---|---|---|---|---|---|
| Name | N | Median | Minimum | Maximum | N | Median | Minimum | Maximum | p-value |
| ER | 38 | 0.009837 | 0.001769 | 0.085101 | 39 | 0.010387 | 0.000000 | 0.064659 | 0.8345 |
| Positivity | | | | | | | | | |
| ER H | 38 | 2.165487 | 0.315269 | 21.777591 | 38 | 2.265046 | 0.103586 | 17.165665 | 0.7514 |
| Score | | | | | | | | | |
| ZEB1 | 38 | 0.004324 | 0.000316 | 0.025044 | 41 | 0.001224 | 0.000221 | 0.028532 | <0.0001** |
| Positivity | | | | | | | | | |
| ZEB H | 38 | 0.931903 | 0.045633 | 5.926867 | 41 | 0.157922 | 0.026349 | 3.927299 | <0.0001** |
| Score | | | | | | | | | |
| FOXA1 | 42 | 0.037941 | 0.010844 | 0.147725 | 47 | 0.021856 | 0.007987 | 0.171964 | 0.0033** |
| Positivity | | | | | | | | | |
| FOXA1 H | 42 | 5.108708 | 1.414022 | 20.066698 | 47 | 3.126083 | 1.033697 | 23.666012 | 0.0031** |
| Score | | | | | | | | | |
| GATA3 | 27 | 0.009031 | 0.001339 | 0.048353 | 32 | 0.018617 | 0.003970 | 0.067257 | 0.0009** |
| Positivity | | | | | | | | | |
| GATA3 H | 27 | 1.656681 | 0.170409 | 10.399523 | 32 | 4.020432 | 0.589316 | 17.773060 | 0.0003** |
| Score | | | | | | | | | |
| MSRB3 | 29 | 0.006854 | 0.002061 | 0.035347 | 26 | 0.006474 | 0.002085 | 0.034037 | 0.4040 |
| Positivity | | | | | | | | | |

TABLE 2
Descriptive Statistics for the Komen dataset

| Characteristic | Frequency | Percent |
|---|---|---|
| Hispanic Ethnicity | | |
| Yes | 0 | 0.0 |
| No | 100 | 100.0 |
| Race | | |
| White | 50 | 50.0 |
| African American | 50 | 50.0 |
| Ashkenazi | | |
| Yes | 2 | 2.0 |
| No | 95 | 96.0 |
| N/A | 2 | 2.0 |
| Smoker? | | |
| Yes | 5 | 5.0 |
| No | 95 | 95.0 |
| Drinker? | | |
| Yes | 60 | 60.0 |
| No | 40 | 40.0 |
| Menstrual Status | | |
| Pre | 85 | 85.0 |
| Post | 9 | 9.0 |
| Uterine Ablation | 6 | 6.0 |
| Ever pregnant? | | |
| Yes | 83 | 83.0 |
| No | 17 | 17.0 |
| Times pregnant | | |
| 1 | 18 | 21.7 |
| 2 | 25 | 30.1 |
| 3 | 18 | 21.7 |
| 4 | 12 | 14.5 |
| 5 | 6 | 7.2 |
| 6 | 2 | 2.4 |
| 7 | 2 | 2.4 |
| Ever breastfed? | | |
| Yes | 56 | 58.3 |
| No | 25 | 26.0 |
| N/A | 15 | 15.6 |
| Hormone | | |
| replacement | | |
| therapy ? | | |
| Yes | 5 | 5.0 |
| No | 94 | 94.0 |
| N/A | 1 | 1.0 |
| Blood family | | |
| relatives with breast | | |
| or ovarian cancer | | |
| No | 90 | 90.0 |
| I don’t know | 10 | 10.0 |

TABLE 3
Descriptive Statistics for the Combined
African American and Caucasian Data of their tumors

| Characteristic | Frequency | Percent |
|---|---|---|
| Ethnicity | | |
| Non-Hispanic | 84 | 98.8 |
| Unknown | 1 | 1.2 |
| Race | | |
| Black or African American | 43 | 50.6 |
| White | 42 | 49.4 |
| Gender | | |
| Female | 84 | 98.8 |
| Unknown | 1 | 1.2 |
| Surgery | | |
| Breast Reduction; | 1 | 1.2 |
| Mastectomy | 1 | 1.2 |
| BSO; Mastectomy/SNB | 1 | 1.2 |
| LND-Axillary | 4 | 4.7 |
| LND-Axillary; Lumpectomy | 4 | 4.7 |
| LND-Axillary; | 23 | 27.1 |
| Lumpectomy/SNB | 9 | 10.6 |
| LND-Axillary; Mastectomy | 5 | 5.9 |
| LND-Axillary; | 5 | 5.9 |
| Mastectomy/SNB | 10 | 11.8 |
| Lumpectomy | 20 | 23.5 |
| Lumpectomy/SNB | 1 | 1.2 |
| Mastectomy | 1 | 1.2 |
| Mastectomy/SNB | | |
| Mastectomy/SNB; | | |
| TVH/BSO | | |
| Mastectomy; TAH/BSO | | |
| Prior Treatments | | |
| Chemo | 16 | 19.8 |
| Chemo; Other Treatment; | 1 | 1.2 |
| RT | 1 | 1.2 |
| Chemo; RT; Surgical | 1 | 1.2 |
| Chemo; Surgical | 2 | 2.5 |
| Combination (NOS) | 50 | 61.7 |
| None | 2 | 2.5 |
| Other Treatment | 1 | 1.2 |
| Other Treatment; RT; | 1 | 1.2 |
| Surgical | 1 | 1.2 |
| Other Treatment; Surgical | 4 | 4.9 |
| RT; Surgical | 1 | 1.2 |
| Surgical | | |
| Unknown | | |

TABLE 4
Tumor versus NAT comparisons Global Comparison of Tumor versus NAT-Unpaired;
wherein the last column shows the two-sided Wilcoxon Test p-values.

| Variable | Tumor | | | | NAT | | | | |
|---|---|---|---|---|---|---|---|---|---|
| Name | N | Median | Minimum | Maximum | N | Median | Minimum | Maximum | p-value |
| ER | 79 | 0.032890 | 0.000251 | 154.458149 | 56 | 0.007863 | 0.000591 | 0.625165 | 0.0095** |
| Positivity | | | | | | | | | |
| ER H | 79 | 6.304881 | 0.033850 | 159.210770 | 56 | 1.786486 | 0.086266 | 159.210770 | 0.0347** |
| Score | | | | | | | | | |
| ZEB1 | 79 | 0.019506 | 0.000427 | 0.108927 | 52 | 0.012089 | 0.001071 | 0.095694 | 0.0177** |
| Positivity | | | | | | | | | |
| ZEB1 H | 79 | 3.427375 | 0.048442 | 23.390940 | 52 | 1.871117 | 0.125566 | 19.138850 | 0.0078** |
| Score | | | | | | | | | |
| FOXA1 | 85 | 0.013202 | 0.001432 | 0.319708 | 83 | 0.019183 | 0.001946 | 0.261425 | 0.1223 |
| Positivity | | | | | | | | | |
| FOXA1 H | 85 | 1.901548 | 0.170627 | 58.676683 | 83 | 2.601070 | 0.265132 | 42.316170 | 0.2443 |
| Score | | | | | | | | | |
| CD8 | 81 | 0.017284 | 0.001530 | 0.105837 | 56 | 0.011015 | 0.001129 | 0.054199 | 0.0040** |
| Positivity | | | | | | | | | |
| CD68 | 83 | 0.008156 | 0.000280 | 0.281922 | 61 | 0.005979 | 0.001434 | 0.159845 | 0.0149** |
| Positivity | | | | | | | | | |
| PD1 | 82 | 0.007431 | 0.001523 | 0.295419 | 67 | 0.007437 | 0.001996 | 0.085643 | 0.6789 |
| Positivity | | | | | | | | | |
| PDL1 | 81 | 0.018127 | 0.003758 | 2.212580 | 62 | 0.022922 | 0.005824 | 0.069939 | 0.0774 |
| Positivity | | | | | | | | | |

**p-value < 0.05

TABLE 5
Differences in Komen Normal Women by BMI; BMI Less than 25 versus BMI of 25
or Greater wherein the last column shows the two-sided Wilcoxon Test p-values.

| Variable | BMI Less than 25 | | | | BMI of 25 or Greater | | | | |
|---|---|---|---|---|---|---|---|---|---|
| Name | N | Median | Minimum | Maximum | N | Median | Minimum | Maximum | p-value |
| ER | 30 | 0.011303 | 0.001563 | 0.044750 | 47 | 0.009762 | 0.000000 | 0.085101 | 0.6685 |
| Positivity | | | | | | | | | |
| ER H | 30 | 2.752854 | 0.276244 | 10.999515 | 46 | 2.165487 | 0.103586 | 21.777591 | 0.7987 |
| Score | | | | | | | | | |
| ZEB1 | 31 | 0.002176 | 0.000221 | 0.012446 | 48 | 0.002248 | 0.000221 | 0.028532 | 0.5402 |
| Positivity | | | | | | | | | |
| ZEB H | 31 | 0.394800 | 0.026349 | 3.053544 | 48 | 0.383249 | 0.026349 | 5.926867 | 0.4046 |
| Score | | | | | | | | | |
| FOXA1 | 36 | 0.028819 | 0.008189 | 0.139036 | 53 | 0.031324 | 0.007987 | 0.171964 | 0.6013 |
| Positivity | | | | | | | | | |
| FOXA1 H | 36 | 3.771841 | 1.243805 | 16.151748 | 53 | 3.834190 | 1.033697 | 23.666012 | 0.7349 |
| Score | | | | | | | | | |

BMI Less than 30 versus BMI of 30 or Greater

| Variable | BMI Less than 30 | | | | BMI of 30 or Greater | | | | |
|---|---|---|---|---|---|---|---|---|---|
| Name | N | Median | Minimum | Maximum | N | Median | Minimum | Maximum | p-value |
| ER | 41 | 0.011262 | 0.001563 | 0.064659 | 36 | 0.009837 | 0.000000 | 0.085101 | 0.4654 |
| Positivity | | | | | | | | | |
| ER H | 41 | 2.517167 | 0.276244 | 17.165665 | 35 | 2.182814 | 0.103586 | 21.777591 | 0.5951 |
| Score | | | | | | | | | |
| ZEB1 | 43 | 0.001632 | 0.000221 | 0.012446 | 36 | 0.002620 | 0.000412 | 0.028532 | 0.0507 |
| Positivity | | | | | | | | | |
| ZEB1 H | 43 | 0.271181 | 0.026349 | 3.053544 | 36 | 0.406620 | 0.050431 | 5.926867 | 0.0365** |
| Score | | | | | | | | | |
| FOXA1 | 50 | 0.030429 | 0.007987 | 0.171964 | 39 | 0.031646 | 0.010677 | 0.147725 | 0.8849 |
| Positivity | | | | | | | | | |
| FOXA1 H | 50 | 3.830976 | 1.033697 | 23.666012 | 39 | 3.827739 | 1.414022 | 20.066698 | 0.8915 |
| Score | | | | | | | | | |

TABLE 6
Paired Differences between Tumor and NAT. The
difference between tumor and NAT was calculated by
subtracting the NAT value from the tumor value.

| | | | | | Two-sided |
|---|---|---|---|---|---|
| | | | | | Wilcoxon |
| Variable | Calculated Difference between Tumor and NAT | | | | Test |
| Name | N | Median | Minimum | Maximum | p-value |

All Patients

| | | | | | |
|---|---|---|---|---|---|
| ZEB1 | 48 | 0.009203 | −0.090741 | 0.083406 | <0.0001** |
| Positivity | | | | | |
| ZEB1 H | 48 | 2.131435 | −18.198427 | 19.015150 | <0.0001** |
| Score | | | | | |
| FOXA1 | 83 | −0.001994 | −0.258264 | 0.296218 | 0.8466 |
| Positivity | | | | | |
| FOXA1 H | 83 | −0.260656 | −41.882805 | 50.727889 | 0.5766 |
| Score | | | | | |
| CD8 | 52 | 0.003980 | −0.011335 | 0.059046 | 0.0012** |
| Positivity | | | | | |
| CD68 | 60 | 0.000850 | −0.146615 | 0.273311 | 0.0203** |
| Positivity | | | | | |
| PD1 | 65 | 0.000047 | −0.074500 | 0.280552 | 0.5627 |
| Positivity | | | | | |
| PDL1 | 60 | −0.003336 | −0.049703 | 0.224029 | 0.1423 |
| Positivity | | | | | |

ER Positive Patients

| | | | | | |
|---|---|---|---|---|---|
| ZEB1 | 30 | 0.009203 | −0.011072 | 0.054486 | 0.0003** |
| Positivity | | | | | |
| ZEB1 H | 30 | 1.928863 | −2.574785 | 12.735212 | 0.0001** |
| Score | | | | | |
| FOXA1 | 49 | 0.005585 | −0.092106 | 0.210113 | 0.0807 |
| Positivity | | | | | |
| FOXA1 H | 49 | 1.445301 | −9.764919 | 50.727889 | 0.0065** |
| Score | | | | | |
| CD8 | 35 | 0.003696 | −0.011335 | 0.049752 | 0.0160** |
| Positivity | | | | | |
| CD68 | 38 | 0.000850 | −0.012214 | 0.044816 | 0.0479** |
| Positivity | | | | | |
| PD1 | 41 | 0.000047 | −0.074500 | 0.022416 | 0.7359 |
| Positivity | | | | | |
| PDL1 | 37 | −0.004180 | −0.049703 | 0.153751 | 0.0660 |
| Positivity | | | | | |

TABLE 7
Differences in Staining of NAT among European Ancestry versus African Ancestry,
wherein the last column is a two-sided Wilcoxon Test p-values.

| Variable | African Ancestry | | | | European | | | | |
|---|---|---|---|---|---|---|---|---|---|
| Name | N | Median | Minimum | Maximum | N | Median | Minimum | Maximum | p-value |
| CD8 | 25 | 0.012709 | 0.003848 | 0.054199 | 31 | 0.009727 | 0.001129 | 0.039320 | 0.0335** |
| Positivity | | | | | | | | | |
| CD68 | 27 | 0.006905 | 0.001434 | 0.159845 | 34 | 0.005630 | 0.001446 | 0.016763 | 0.2667 |
| Positivity | | | | | | | | | |
| PD1 | 34 | 0.010032 | 0.002080 | 0.050013 | 33 | 0.004972 | 0.001996 | 0.085643 | 0.0002** |
| Positivity | | | | | | | | | |
| PDL1 | 28 | 0.017939 | 0.005824 | 0.037796 | 34 | 0.027684 | 0.007620 | 0.069939 | 0.0003** |
| Positivity | | | | | | | | | |

### Example 9

ERα status in tumors influence differences between NATs and tumors: Although we observed differences in ZEB1, GATA3, and ERα expression between NATs and tumors (FIGS. 1D, 2D and 3D), interpretation of these data is difficult because of several different subtypes of breast cancer, particularly ERα-positive and ERα-negative. To determine whether ERα-positive and ERα-negative tumors have distinct effects on NATs, FOXA1, GATA3, and ZEB1 expression data in NATs and tumors were subdivided based on ERα status of the tumor and reanalyzed. ZEB1-positivity and H-scores were increased in ERα-positive but not ERα-negative tumors compared to NATs (Table 4). Despite small sample size, these differences were noted in only EA women with ERα+ breast cancers (Table 4). With respect to FOXA1, H-score but not positivity was marginally higher in ERα-positive of EA women (Table 4). ERα-negative tumors of EA but not AA women showed a significant decline in both positivity and H-score of FOXA1 compared to NATs (Table 4). ERα-positive tumors but not ERα-negative tumors showed further increase in GATA3 positivity and H-scores in EA women, which further confirms the role of GATA3 in hormonal regulation of breast cancer (FIG. 8C and Table 4). When the analyses was done with paired NAT-tumors, the above noted differences between NATs and tumors in ZEB1, GATA3, and FOXA1 levels remained significant, although sample size was too small to subdivide samples based on genetic ancestry (Table 6).

### Example 10

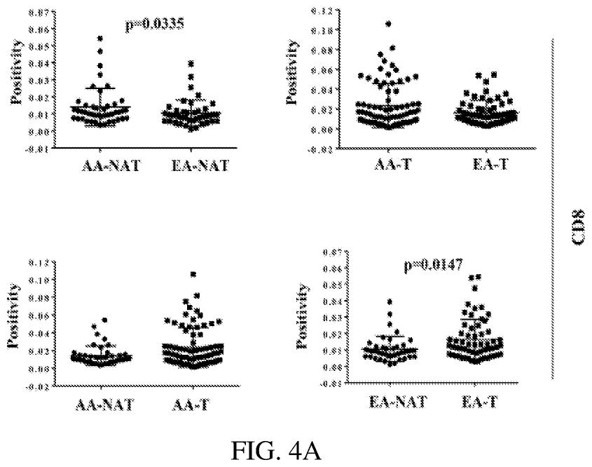

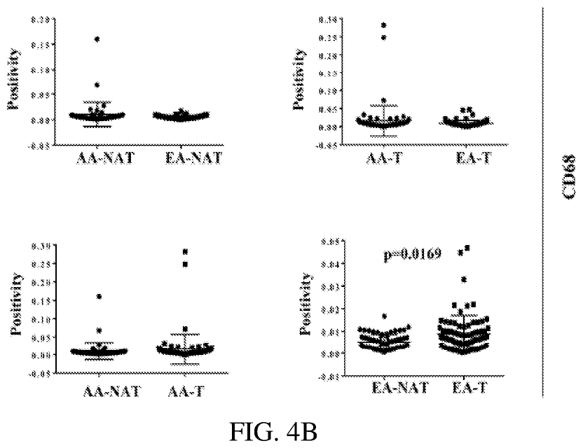

NATs of AA and EA women show differing levels of CD8, PD1 and PDL1+ cells. Results thus far point to the pro-inflammatory state of NATs based on known link between ZEB1 and inflammatory cytokines. To address this further, we stained the above TMAs with CD8 for T cells, CD68 for macrophages, and PD1 for immune cells. We also examined epithelial/tumor cells for PDL1. All staining was done in a CLIA-certified lab with FDA-approved antibodies. In KTB-normal TMAs, there was no staining with CD8 and CD68 in either the AA or EA TMAs. There were less than 1% of the lymphocytes and macrophages stained and these were considered negative. The same negativity was obtained with PD1 and PDL1 immunostains. Therefore, we analyzed staining results between NATs of AA and EA women and NATs and tumors. CD8 immunostaining was localized to the inflammatory cells (T lymphocytes) and not in the tumor cells in the breast cancer cores. No background reactivity was observed in all cases. NATs of AA women showed statistically significantly higher CD8 positivity compared to EA women (FIG. 4A and Table 7). The tumors in EA had more CD8 immunostaining compared to corresponding NATs but such differences were not seen in the AA women.

CD68 staining was localized to macrophages in the breast cancer cores. CD68 had lower positivity compared to CD8 by both visual and the Aperio positive pixel reads. CD68 positivity was higher in tumors compared to their NATs (p=0.02) in EA but not in AA women (FIG. 4B).

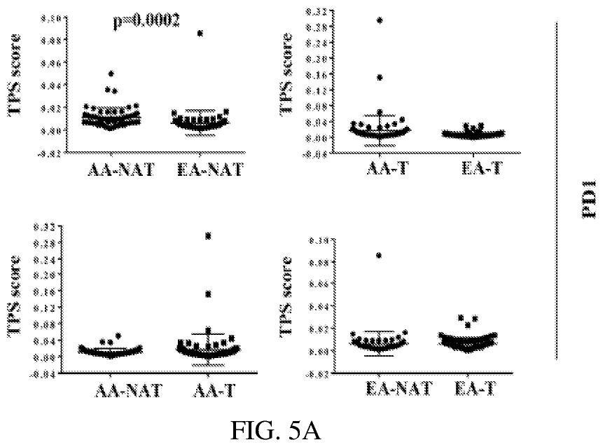

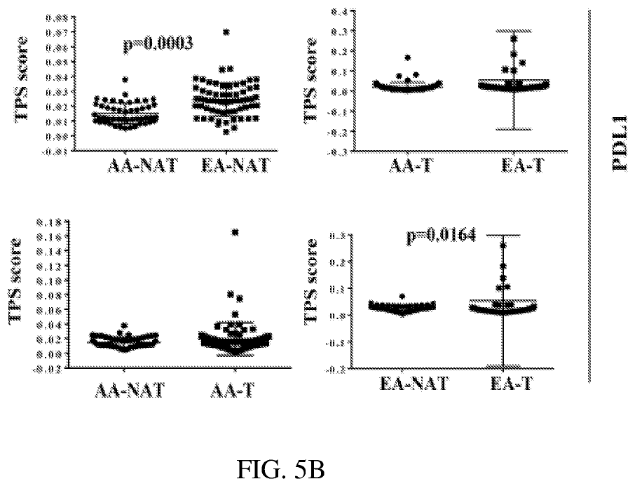

PD1 immunostaining was localized to immune cells only and no background staining was observed. There was no staining of tumor cells. NATs of AA women contained significantly higher PD1+ cells, similar to CD8+ cells, compared to NATs of EA women (FIG. 5A and Table 6). PD1 staining did not show any differences between NATs and tumors in both groups (Table 4).

PDL1 immunostaining was seen localized in the tumor cell cytoplasm and cell membrane. In a few EA cases, only lymphocytes were stained. PDL1 staining of NATs of AA was significantly lower than EA cases (FIG. 5B and Table 7). Although PDL1 staining did not differ between NATs and tumors in case of AA women, its levels were marginally lower in ERα+ tumors but not ERα− tumors compared to NATs in case of EA women (FIG. 5 and Table 4). It is interesting that PD1 and PDL1 staining scores in NATs of AA is reverse of the patterns in EA women. Accordingly, the immune environment in NATs is different from that in KTB-Normal with further differences between NATs and tumors, which showed variations based on genetic ancestry.

### Example 11

Recent studies have shown cancer-induced field effect influencing gene expression pattern in histologically normal tissues surrounding cancer. These observations raise a concern that the use of NATs as a “normal” control may introduce bias into such analysis. There is also an opportunity to develop assays based on cancer-induced field effect on NATs as early markers of breast cancer. However, recent discovery of inter-individual differences in gene expression pattern due to single nucleotide polymorphisms (SNPs) in gene regulatory regions and ancestry-dependent enrichment of SNPs with breast cancer protective or elevated risk characteristics necessitate the use of ancestry-matched normal control samples from healthy women to develop molecular features of tumor adjacent normal as early markers. Ethnicity contributing to inter-individual differences in normal biology is just beginning to be explored, as evident from a recent study that demonstrated distinct gut microbiota in different ethnic groups with shared geography. Furthermore, genetic ancestry has been shown to influence mutation patterns in cancer.

Resources available at the Komen Normal Tissue Bank, particularly ancestry-mapped >5000 breast tissues from healthy women, enable these factors to be taken into consideration for developing molecular features of NATs as cancer detection markers. Utilizing a small fraction of those tissues, evidence for ancestry-dependent differences in the number of ZEB1+ and GATA3+ cells in the normal breast tissue as well as cancer-induced field effects on ZEB1, GATA3, and FOXA1+ cells in NATs have been demonstrated.

Recently discovered functions of ZEB1 have raised considerable interest on this molecule in the oncology field. The regulatory regions of this gene remain in a bivalent state, enabling the regulatory regions to respond readily to the tumor microenvironment and increase breast cancer plasticity and tumorigenicity. Another study showed elevated ZEB1 expression in normal breast stem cells and it functionally protects stem cells from p53-mediated cell death in response to oncogene activation-induced DNA damage and promotes tumorigenicity with limited genomic instability. It was also reported that ZEB1 is expressed in both tumor and stromal cells of the breast tissue. ZEB1 directly increases the expression of pro-inflammatory cytokines such as IL-6 and IL-8 and it promotes vascular mimicry of breast cancer cells by remodeling extracellular matrix. Applicant had previously demonstrated that cytokines such as tumor necrosis factor induce the expression of ZEB1. These observations along with current unique observations of genetic ancestry-dependent differences in ZEB1-positive cells in the normal breast tissue, elevated number of ZEB1+ cells in the NATs compared to their healthy counterparts of women of European ancestry and its localization outside the ductal structures raise several questions about the function of ZEB1+ cells in the normal NATs breast tissue. Cytokeratin-positive, PROCR+/EpCAM− cells of the normal breast tissue, which are enriched in the normal breast tissue of AA women compared to EA women, have been previously shown to express 50-fold higher levels of ZEB1 compared to cytokeratin-positive, PROCR−/EpCAM+ cells of the breast tissue. Thus, we suspect that ZEB1+ cells in the normal breast tissue correspond to PROCR+/EpCAM− cells and cancer-induced field effect leads to their proliferation in the breast tissue of EA women. Signaling pathways leading to their proliferation are unknown but the Wnt pathway is the prime suspect as it is activated in cells surrounding cancer due to altered DNA methylation. In this respect, Wnt and ZEB1 constitute a reciprocal feed-forward signaling loop where ZEB1 enhances TCF4/β-Catenin-mediated transcription and Wnt signaling converts ZEB1 from a transcription repressor to an activator.

In contrast to stemness-associated ZEB1, FOXA1, which is expressed predominantly in differentiated luminal cells, showed an opposite pattern in AA women. While the normal breast tissue of AA women had higher number of FOXA1+ cells compared to EA women, there is a decline in FOXA1+ cells in NATs as a consequence of cancer induced field effect observed only in AA women. How tumors cause down regulation of FOXA1 in NATs is unknown but could involve inflammatory cytokines, as cytokine inducible transcription repressors such as TWIST1 repress FOXA1 expression. In this regard, applicant observed genetic ancestry-dependent differences in the levels of immune cells in NATs; NATs of AA women contained elevated number of CD8+ T cells and PD1+ immune cells compared to NATs of EA women. In addition, FOXA1 regulatory regions are highly susceptible for DNA methylation and transcriptional repression, particularly in the context of BRCA1 deficiency. Furthermore, ERα-negative tumors in AA women show elevated FOXA1 DNA methylation compared to ERα-negative tumors of EA women. Recent studies have also demonstrated racial differences in plasma levels of cytokines with CCL2, CCL11, IL4, and IL10 being higher in women of EA and IL1RA and IFNα2 being higher in AA women.

Differential expression of GATA3 in the normal breast tissue of AA and EA women is intriguing, as GATA3 is one of the major signaling molecules required for hormonal response and differentiation of normal breast epithelial cells. Our results suggest that hormonal- and differentiation-signaling networks show genetic ancestry-dependent differences and it is likely that ERα:GATA3-dependent transcriptional program is more active in the normal breast tissue of EA compared to AA women.

Collectively, data presented herein suggest the need to consider the following aspects for cancer biomarker discovery: 1) NATs are molecularly abnormal and thus not suitable as controls; 2) These abnormalities can be detected only when relevant true normal breast tissues are used as controls and differences in normal gene expression attributable to genetic ancestry are taken into consideration; 3) ZEB1 shows unique expression pattern in normal breast tissue and is a suitable biomarker of breast cancer initiation of women of European Ancestry; and 4) genetic ancestry has an influence on the immune environment of tumors as well as NATs.

### Example 12

AA women genetically identified as duffy-null experience more aggressive breast cancer. To investigate the role of hyperactive c-MET signaling in breast tumorigenesis under the duffy-null/heterozygous background, we can compare growth and invasive properties of transformed and non-transformed breast epithelial cells from duffy-null, duffy-heterozygous, and duffy-wild type AA women under basal and CCL2/CXCL8 treated conditions. Downstream signaling by these chemokines can be investigated using phosphoproteomics.

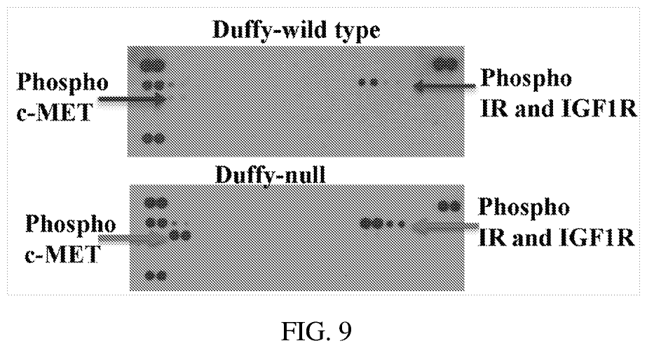

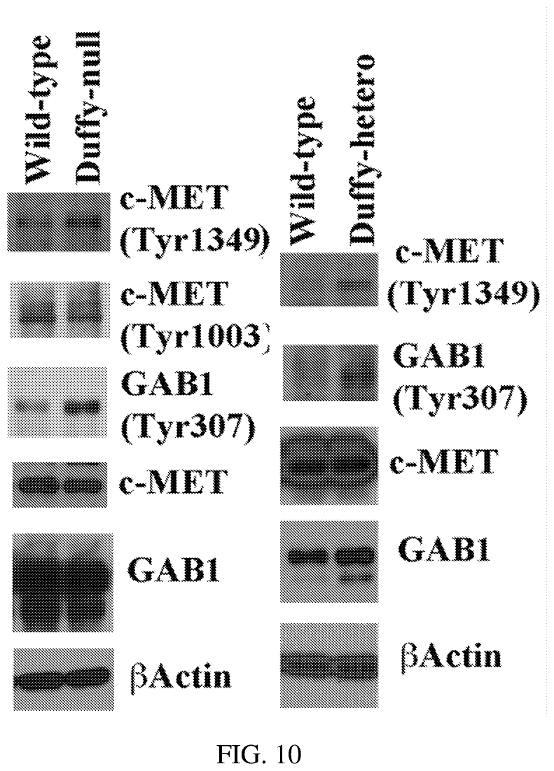

Genotyping revealed ˜40% of AA women who donated breast tissue to the tissue bank are duffy-null or heterozygous. The ACKR1/DARC receptor, which is either not expressed or expressed at lower levels in these 40% of AA women, serves as a decoy receptor for chemokines CCL2 and CXCL8. In the presence of ACKR1/DARC in epithelial cells, signaling by these cytokines can be lower compared to cells that do not or carry lower levels of these receptors. Both CCL2 and CXCL8 are linked to aggressive breast cancer tumor biology with CXCL8 playing a specific role in anti-estrogen and chemotherapeutic response. For example, CXCL8 levels remain high in post-neoadjuvant treatment samples that have residual disease. Similarly, CCL2 is considered as a therapeutic target for ERα+ breast cancer. Thus, breast cancers in duffy-null or duffy-heterozygous carriers may be unduly influenced by these chemokines compared to breast cancers in duffy-wild type women. Our studies indicate that breast epithelial cells in duffy-null carriers have higher levels of activated c-MET. See FIGS. 9 and 10. Activated c-MET itself has been shown to induce secretion of CXCL8 and promote brain metastasis of breast cancer. Thus, duffy-null or duffy-heterozygous phenotype may create a hyperactive cMET-CXCL8 feed-forward loop that confers aggressive growth characteristics including metastasis.

1-4. (canceled)

5. A method of analyzing potential cancer or pre-cancer biopsy samples of patients of European Ancestry or African Ancestry, said method comprising:
obtaining a potential cancer or pre-cancer biopsy sample recovered from patient tissues;
categorizing said sample based on ancestry mapping of the patient source of said sample; and
quantifying the level of expression in said categorized sample of a protein marker selected from a group consisting of ZEB1, FOXA1, and GATA3, or a combination thereof.

6. The method of claim 5 wherein the quantification step comprises
i) quantifying the number of cells expressing ZEB1, and the number of cells expressing FOXA1; or
ii) quantifying the number of cells expressing ZEB1, and the number of cells expressing GATA3; or
iii) quantifying the number of cells expressing ZEB1, the number of cells expressing FOXA1, and the number of cells expressing GATA3.

7. (canceled)

8. The method of claim 6, wherein the quantification step comprises quantifying the level of expression of ZEB1 and FOXA1 in said categorized sample.

9. The method of claim 8, wherein the biopsy sample is provided by a woman of African Ancestry.

10. The method of claim 7, wherein the biopsy sample is provided by a woman of European Ancestry.

11. The method of claim 5 further comprising screening said biopsy sample for duffy-null alleles.

12. The method of claim 5 wherein the biopsy sample is recovered from human female breast tissue.

13. The method of claim 5 further comprising quantifying the expression levels of phospho-c-MET.

14. (canceled)

15. (canceled)

16. An improved method of treating cancer in a patient of European Ancestry or African Ancestry, said method comprising the steps of
obtaining a tissue biopsy sample identified as being cancer by radiological techniques and categorized as being from a patient of European Ancestry or African Ancestry;
analyzing said sample for an increased level of ZEB1+ cells and decreased levels of FOXA1+ cells compared to a relevant true normal sample or relevant true normal population data;
identifying patients of European Ancestry and having an increased level of ZEB1+ cells;
identifying patients of African Ancestry and having decreased levels of FOXA1+ cells; and
treating said identified patients of European Ancestry and patients of African Ancestry using anti-cancer therapies.

17. The method of claim 16 wherein the patient is a woman and the biopsy sample is recovered from breast tissue.

18. An improved method of analyzing a pre-cancer or cancerous biopsy in a patient who is duffy-null/heterozygous, said method comprising the steps of
analyzing a tissue biopsy sample obtained from said patient and identified as cancerous or pre-cancerous using radiology techniques; and
quantifying the levels of phospho-c-MET in the patient sample.

19. The method of claim 18, further comprising quantifying the levels of ZEB1+ cells in said biopsy sample.

20. (canceled)

21. A kit for detecting and quantitating the relative number of ZEB1+ cells and FOXA1+ cells in a tissue sample recovered from a patient, said kit comprising
a reagent for the detection of ZEB1+ cells; and
a reagent for the detection of FOXA1+ cells.

22. The kit of claim 21 wherein the kit comprises an antibody specific for ZEB1 and an antibody specific for FAOXA1.

23. The kit claim of 21 further comprising a reagent for detecting GATA3.

24. The kit claim of 21 further comprising a reagent for detecting phospho-c-MET.

25. An improved method of analyzing a pre-cancerous or cancerous biopsy sample from a patient of European Ancestry, said method comprising the steps of
analyzing the biopsy sample identified as pre-cancerous or cancerous using radiology techniques; and
quantifying the number of GATA3+ cells.

26. The method of claim 25 further comprising quantifying the number of ZEB1+ cells.

27. (canceled)
